SEQUENCE LISTING (12 sequences)

SEQ ID NO: 1 - DNA, 25 bp, Artificial Sequence
  Synthetic Probe for P.pachyrhizi
ccaaaaggtacacctgtttgagtca

SEQ ID NO: 2 - DNA, 20 bp, Artificial Sequence
  Synthetic Probe for GBS sip
cccgaataacccggatattt

SEQ ID NO: 3 - DNA, 20 bp, Artificial Sequence
  Synthetic Sip forward primer for GBS
taccgcgaaagagcagattt

SEQ ID NO: 4 - DNA, 20 bp, Artificial Sequence
  Synthetic Backward primer for GBS sip
aagcagtcgagtgcctcatt

SEQ ID NO: 5 - DNA, 21 bp, Artificial Sequence
  Synthetic Forward primer for GBS cfb
tgatcaagcttttgagccatt

SEQ ID NO: 6 - DNA, 25 bp, Artificial Sequence
  Synthetic Probe for GBS cfb
ctcaacagtgaactagtcgtacatg

SEQ ID NO: 7 - DNA, 27 bp, Artificial Sequence
  Synthetic Backward primer for GBS cfb
tgatgtatctatctggaactctagtgg

SEQ ID NO: 8 - protein, 15 aa, Artificial Sequence
  Synthetic Trypsin substrate
RFKLRRFARRRVRRA

SEQ ID NO: 9 - protein, 26 aa, Artificial Sequence
  Synthetic Rennin substrate
RPFHLLVYSEAEAVFVFVFLFVFVFL

SEQ ID NO: 10 - protein, 25 aa, Artificial Sequence
  CDS 3: Synthetic HIV-1 protease substrate
SQNYPIVQEAEAVFVFVFLFVFVFL

SEQ ID NO: 11 - protein, 25 aa, Artificial Sequence
  CDS 7: Synthetic Factor IIa substrate
VPRSFRNAEAEAVFVFVFLFVFVFL

SEQ ID NO: 12 - protein, 25 aa, Artificial Sequence
  CDS 8: Synthetic Factor Xa substrate
IEGRTSENEAEAVFVFVFLFVFVFL

(19) United States
(12) Patent Application Publication
(10) Pub. No.: US 2006/0199241 A1
(43) Pub. Date: Sep. 7, 2006

(54) Electrochemically and optically monitoring cleaving enzyme activity

(76) Inventors: Hyoungsik Yim, Ann Arbor, MI (US); Mark E. Meyerhoff, Ann Arbor, MI (US); Dongxuan Shen, Ann Arbor, MI (US); Sangyeul Hwang, Ann Arbor, MI (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 11/264,288

(22) Filed: Nov. 1, 2005

Related U.S. Application Data
(63) Continuation-in-part of application No. 11/070,568, filed on Mar. 2, 2005.

(51) Int. Cl. C12Q 1/37; C07K 14/47; C07K 14/80

(52) U.S. Cl. 435/23; 530/350; 530/409

(57) ABSTRACT

A marker molecule for monitoring cleaving enzyme activity is disclosed. The marker molecule includes a protein, a peptide, or an oligonucleotide. A co-factor is conjugated to the protein, the peptide, or the oligonucleotide, thereby forming a co-factor labeled protein, a co-factor labeled peptide, or a co-factor labeled oligonucleotide. The co-factor is adapted to produce an enzymatic signal that is at least one of electrochemically and optically detectable.

### CROSS REFERENCE TO RELATED APPLICATIONS

This application is a continuation-in-part of U.S. application Ser. No. 11/070,568, filed Mar. 2, 2005.

### BACKGROUND

The present disclosure relates generally to electrochemically and/or optically monitoring cleaving enzyme activity, and more particularly to the electrochemical and/or optical detection of enzyme (polymerase, DNase, and protease, etc.) activity using a specially designed co-factor labeled protein, peptide, or oligonucleotide.

Genetic testing and enzyme-based assays have the potential for use in a variety of applications, ranging from genetic diagnostics of human diseases to detection of trace levels of pathogens in food products. Currently, more than 800 diseases can be diagnosed by proteomics and molecular biology analysis of nucleic acid sequences. It is likely that additional tests will be developed as further proteomic and genetic information becomes available. Protein and DNA diagnostic devices enable clinicians to efficiently detect the presence of a whole array of proteomic and genetic based diseases, including, for example, AIDS, Alzheimer's, and various forms of cancer.

The rising use of protein and/or DNA diagnostic testing devices has produced a need for low-cost, highly portable protein and/or DNA detection devices (for example, a glucometer-type “lab-on-a-chip” device) for use in various markets including health care, agriculture, food testing and bio-defense. Generally, it would be desirable that any new protein and/or DNA diagnostic devices integrate several functional analysis components within the same platform. Further, it would be desirable that such devices be reliable, inexpensive, and able to simplify the monitoring of EA (cleaving enzyme activity) and PCR (polymerase chain reaction).

### SUMMARY

A marker molecule for monitoring cleaving enzyme activity is disclosed. The marker molecule includes a protein, a peptide, or an oligonucleotide. A co-factor is conjugated to the protein, the peptide, or the oligonucleotide, thereby forming a co-factor labeled protein, a co-factor labeled peptide, or a co-factor labeled oligonucleotide. The co-factor is adapted to produce an enzymatic signal that is electrochemically and/or optically detectable.

A method of monitoring cleaving enzyme activity in a sample is also disclosed. The method includes exposing a co-factor labeled protein, peptide, or oligonucleotide to cleaving activity. The co-factor labeled protein, peptide, or oligonucleotide includes a protein, peptide, or oligonucleotide and a co-factor conjugated to the protein, peptide, or nucleotide. This exposure releases a fragment including the co-factor. The fragment is then combined with an apo-enzyme. Combining the fragment having the co-factor with the apo-enzyme produces an enzymatic signal that is electrochemically and/or optically detectable. The enzymatic signal, which is electrochemically and/or optically detectable, confers detection of cleaving activity.

### BRIEF DESCRIPTION OF THE DRAWINGS

Objects, features and advantages of embodiments of the present invention will become apparent by reference to the following detailed description and drawings, in which:

FIG. 1 is a schematic flow diagram illustrating an embodiment of making an embodiment of a prosthetic group (PQQ) labeled oligonucleotide;

FIG. 2A is a schematic view of an embodiment of a method of detecting DNA;

FIG. 2B is a graph depicting optical PCR detection for Group B Streptococcous cfb gene;

FIG. 3A is a schematic flow diagram illustrating an embodiment of making an embodiment of a prosthetic group (PQQ) labeled protein (e.g., protamine);

FIG. 3B is a schematic flow diagram illustrating another embodiment of making an embodiment of a prosthetic group (PQQ) labeled polypeptide;

FIG. 4 is an exploded, partially schematic view of a specific example embodiment of a method of monitoring peptide cleaving enzyme activity;

FIG. 5 is a schematic view of specific example embodiments of PQQ labeled probes;

FIG. 6 is a graph depicting real time trypsin enzyme activity for cleaving protamine labeled with PQQ using DCIP as the redox indicator;

FIG. 7 is a graph depicting real time trypsin activity for cleaving trypsin enzyme labeled with PQQ using DCIP as the redox indicator;

FIG. 8 is a graph depicting GDH enzyme activity for various concentrations of PQQ using DCIP as the redox indicator; and

FIG. 9 is a graph depicting real time DNase activity for cleaving a PQQ labeled oligonucleotide using DCIP as the redox indicator.

### DETAILED DESCRIPTION

Embodiment(s) disclosed herein advantageously combine a marker molecule (e.g. a co-factor labeled protein, peptide, or nucleotide) and the production of an enzyme amplified electrochemically and/or optically detectable signal, both of which may be incorporated into a DNA diagnostic device or an EA monitoring device. This combination provides an enzyme-based electrochemical and/or optical method to detect DNA amplified via polymerase chain reaction (PCR) or to monitor cleaving enzyme activity (EA). The cleaving enzyme activity may be used to monitor the anticoagulation effect of an anticoagulation reagent (e.g. heparin), to measure activated clotting time (ACT), to measure activated partial thromboplastin time (aPTT), to measure thrombin time (TT), and/or the like. It is to be understood that embodiment(s) of the marker molecule may be integrated with, for example, a litmus paper-type strip sensing system, a multi-well plate with a plate reader, or a flow-through system with a visible spectrometer for end-point PCR detection and/or EA optical monitoring.

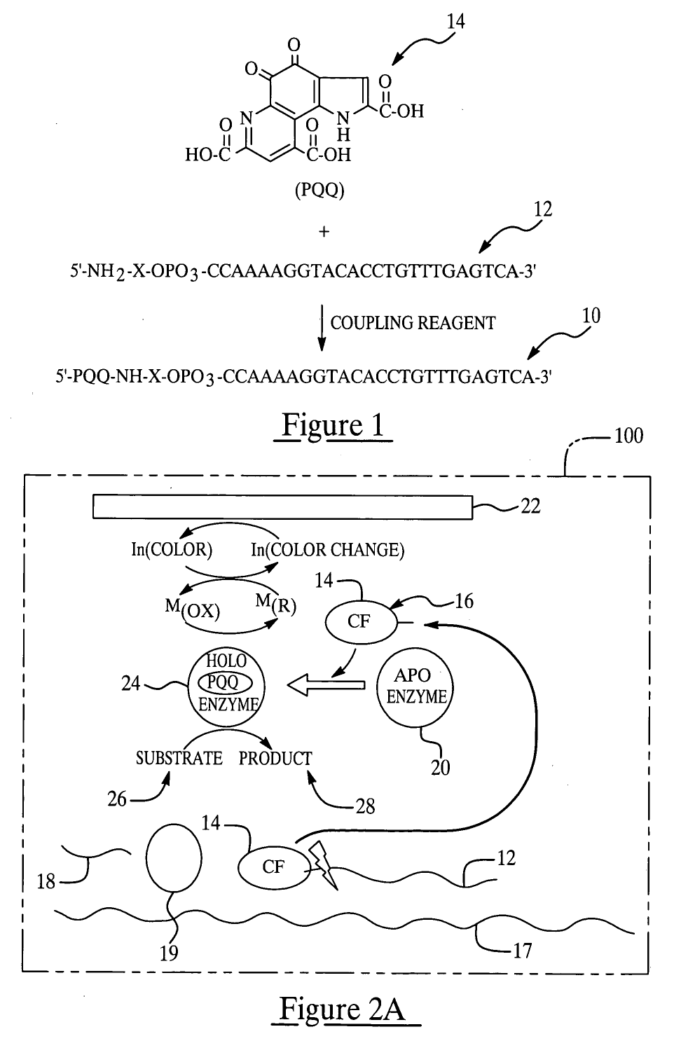

Referring now to FIG. 1, an embodiment of making a labeled oligonucleotide 10 (i.e. marker molecule) is schematically depicted. Generally, embodiments of the labeled oligonucleotide 10 include a site-specific sequence 12 labeled with a co-factor (CF) 14. The co-factor (CF) 14 may be conjugated at any spot along the site-specific sequence 12. In non-limitative example embodiments, the co-factor (CF) 14 may be attached to the 5′ end, the 3′ end, and/or anywhere between the two ends.

In an alternate embodiment, the marker molecule 10 is a labeled peptide or a labeled protein. Generally, these embodiments include a co-factor conjugated to a selected protein or peptide.

In the non-limitative example shown in FIG. 1, the sequence 12 is a 5′amine-derivatized oligonucleotide. It is to be understood that a variety of 5′amine-derivatives oligonucleotides may be prepared by a phosphoramidite method. Further, any amine-derivatized phosphoramidite may be used in this embodiment. As depicted, the amine-derivatized oligonucleotide includes an amine modified linker, X, which may be, but is not limited to —(CH₂)ₙ—, —(CH₂)ₒO(CH₂)ₚ, and/or —(CH₂)_{q}S(CH₂)ᵣ, wherein n=2-12 and o,p,q and/or r=2-6. The marker molecule 10 may be prepared by coupling the co-factor (CF) 14 (PQQ) with the 5′amine-derivatized oligonucleotide using common coupling reagents, such as 1-(3-dimethylaminopropyl)-3-ethylcarbodiimide (EDC or EDAC), and an appropriate base, such as N-methylmorpholine.

In embodiment(s) of the method, the co-factor (CF) 14 is adapted to produce an enzymatic signal that is electrochemically and/or optically detectable. As used herein, the term “produce” means indirectly or directly generating the enzymatic signal. In a non-limitative example, indirectly producing includes binding the co-factor (CF) 14 to an apo-enzyme to form an activated enzyme that is capable of catalyzing a reaction that results in an electrochemically and/or optically detectable enzymatic signal.

Generally, as described in more detail hereinbelow, the co-factor (CF) 14 portion of the marker molecule 10 binds with an apo-enzyme. Non-limitative examples of the co-factor (CF) 14 include prosthetic groups (organic and covalently bound to an enzyme), co-enzymes (organic and non-covalently bound to an enzyme), and metal-ion activators. Non-limitative examples of metal ion activators include iron, copper, manganese, magnesium, zinc, and the like, and combinations thereof. Specific non-limitative examples of co-factors 14 include pyrroloquinoline quinine (PQQ), flavin adenine dinucleotide (FAD), nicotinamide adenine dinucleotide phosphate (NADP), or heme. Other suitable co-factors 14, specifically those that may be used in place of PQQ include, but are not limited to phenazine methosulfate, phenazine ethosulfate, naphthoquinone, derivatives thereof, benzoquinone, derivatives thereof, fluorescein, derivatives thereof, thionine, resazurin, 2,6-dichlorphenoleindophenole, orthoquinone, and derivatives thereof, and the like. It is to be understood that any fused ring compound which may be reduced by accepting 2 electrons and 2 hydrogens from a substrate (e.g. glucose) may be used as the co-factor 14. Non-limiting examples of orthoquinone (A) and its derivatives (B through I) are depicted. The derivatives have either 5- and/or 6-membered rings, 2 or 3 of which are fused together. In the non-limitative examples shown below, X═—(CH₂)ₙ, —OCH₂(CH₂)ₙ, —NHCH₂(CH₂)ₙ, and/or —SCH₂(CH₂)ₙ, wherein n=0-6; Y═—CO₂H, SO₃H, SO₄H, PO₃H and/or PO₄H; Z=CH₂, NH, O, or S (with saturated bond) or CH and/or N (with unsaturated bond); and k=1-4.

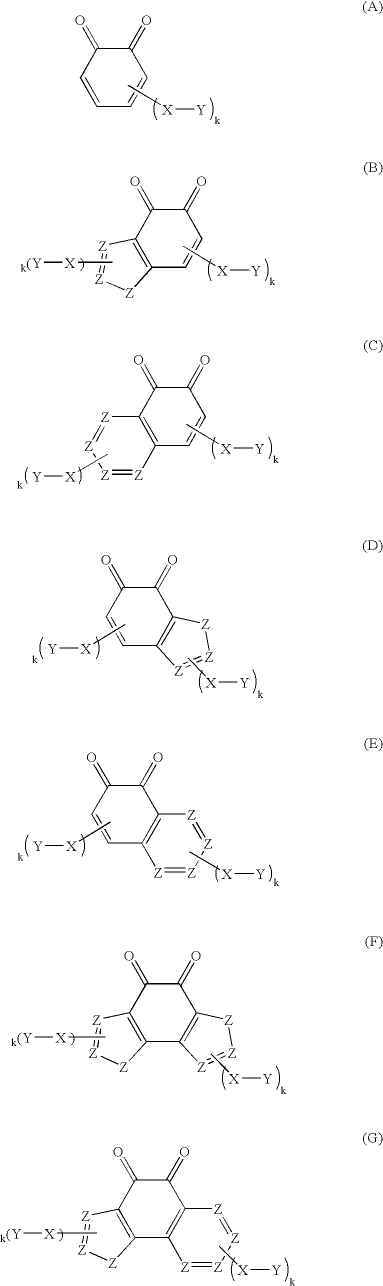

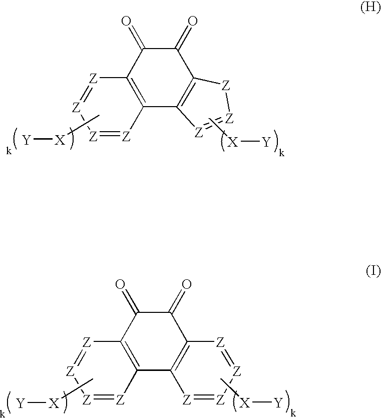

The non-limitative example shown in FIG. 1 depicts pyrroloquinoline quinone (PQQ) as the co-factor (CF) 14, and 5′-NH₂—X—OPO₃-CCAAAAGGTACACCTGTTTGAGTCA-3′ as the site-specific sequence 12. The PQQ is conjugated to the amino group of the sequence 12. The non-limitative example of marker molecule 10 shown in FIG. 1 is capable of detecting target DNA from P. pachyrhizi. It is to be understood, however, that the marker molecule 10 may be made complementary to any target DNA (e.g. Group B Streptococcous (GBS) sip or cfb gene). Further, the marker molecule 10 may be designed to hybridize or anneal to its complementary single strand DNA sequence within an amplicon domain defined by a pair of primer oligonucleotides or between forward and reverse primers.

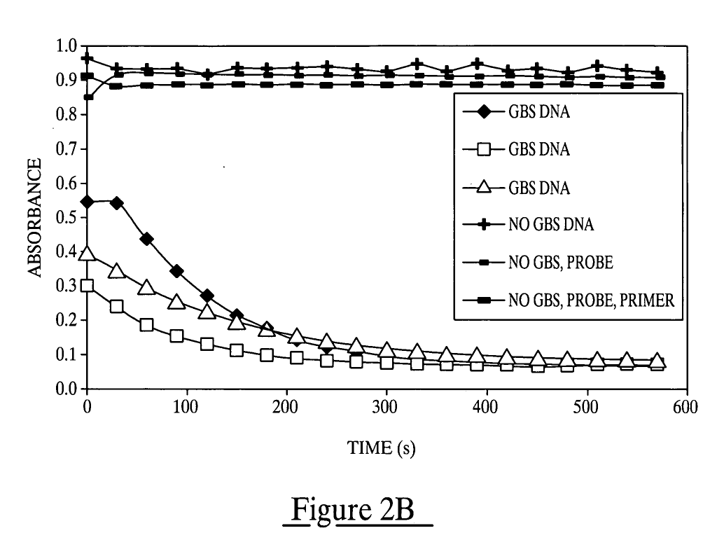

Referring now to FIG. 2A, an embodiment of the device 100 and the method of detecting target DNA in a sample using an embodiment of the labeled oligonucleotide 10 is schematically depicted. The device 100 may be an electrochemical diagnostic device and/or an optical diagnostic device. Generally, an electrochemical diagnostic device 100 includes an electrode 22 with an apo-enzyme 20 immobilized to a surface thereof, and a reaction mixture including molecule marker (or labeled oligonucleotide) 10 in electrochemical contact with the electrode 22, after the amplification process occurs. An optical diagnostic device 100 includes a multi (e.g. 96)-well plate (or a litmus paper-type strip) 22 with a plate reader and a reaction mixture including an apo-enzyme and a molecule marker (or labeled oligonucleotide) 10, after the amplification process occurs.

Embodiment(s) of the method integrate DNA amplification processes (non-limitative examples of which include real-time and end-point PCR) with enzymatic signal amplification. More specifically, PCR-dependent exonuclease activity can trigger enzymatic generation or amplification of a measurable electrochemical and/or optical enzymatic signal. The enzymatic signal(s) may be optically detectable via absorbance change, fluorescence, visual color change, electrochemistry, densitometry, and combinations thereof. The enzymatic signal may be electrochemically detectable via voltammetry, amperometry, coulometry, potentiometry, conductivity, tensammetry, impedance measurements, and/or combinations thereof.

Embodiment(s) of the method generally include performing a DNA amplification process on a sample 17, exposing an embodiment of the probe 10 to exonuclease activity, combining a co-factor (CF) 14 probe fragment 16 to an apo-enzyme 20, and electrochemically and/or optically detecting an enzymatic signal that results from the combination of the co-factor (CF) 14 with the apo-enzyme 20. It is to be understood that these steps may be performed substantially simultaneously or sequentially.

Non-limitative examples of the DNA amplification processes include end-point PCR, real-time PCR, PCR-free amplification (using a PCR mixture without thermocycles), rolling cycle amplification (RCA), isothermal based amplification methods, and thermocycling based amplification methods. The processes may include a reaction (or PCR) mixture and/or sample formulated such that it is compatible with desired chemistries for enzymatic signal amplification and electrochemical and/or optical detection. Such a formulation may include, but is not limited to the following: substrate(s) (a non-limitative example of which is glucose), probes 10, buffers (non-limitative examples of which include Tris, HEPES, phosphate, and the like), mediators (non-limitative examples of which include ferricyanide, ferrocene derivatives, phenazine methosulfate (PMS), Ru(III) complexes, ubiquinone (Q₀), Os complexes, and the like), stabilizers (non-limitative examples of which include CaCl₂, MgCl₂, and the like), redox indicators (non-limitative examples of which include dichloroindolephenol (DCIP), resazurin, thionine, and the like), enzyme thermal stabilizers, barriers, oligo binders, and/or mixtures thereof.

As depicted in FIG. 2A, the intact labeled oligonucleotide 10 includes the sequence 12 having a co-enzyme as the co-factor (CF) 14 conjugated thereto. During the DNA amplification process, 5′→3′ exonuclease activity of DNA polymerase enzyme 19 results in the hydrolysis of the probe 10. The size and locations of DNA being amplified (amplicon) is determined, at least in part, by a pair of primer oligonucleotides (arbitrarily designated forward and reverse primers) which are complimentary and hybridize specifically to double stranded template DNA that is denatured by thermal cycles in the amplification process.

Referring still to FIG. 2A, forward primer 18 is not attached to the DNA polymerase enzyme 19. It is to be understood that the forward primer 18 in the mix hybridizes to a complimentary region on the target DNA 17. In this embodiment, the DNA polymerase enzyme 19 has a binding site that recognizes a 3′ terminus structure of the forward primer 18. The DNA polymerase enzyme 19 then extends the primer 18 by filling in complimentary bases over the single stranded template.

The hydrolysis of the labeled nucleotide 10 releases (as depicted by the lightening bolt) a fragment 16 containing the co-factor (CF) 14. The co-factor (CF) 14 (e.g. a co-enzyme, prosthetic group, or metal-ion activator) of the fragment 16 may then combine with and activate an apo-enzyme 20 immobilized on the surface of a test strip 22 (a non-limitative example of which includes a PCR test strip), or multi-well plate 22, or an electrode 22. It is to be understood that the apo-enzyme 20 may also be present in solution (which may be disposed in a well of multi-well plate 22) when the assay is homogeneous. The combination of the fragment 16 and the apo-enzyme 20 forms a holo-enzyme 24, which is capable of catalyzing a reaction that converts a predetermined substrate 26 in the sample to a product 28 plus free electrons. These free electrons may reduce a mediator M_{(R)}, which is subsequently re-oxidized M(ox) by a redox indicator (In(color)) (a non-limitative example of which includes a dye) that results in the color change of the redox indicator (In(color change)). The free electrons may also reduce a co-substrate (i.e. a reactant that is transiently associated with the enzyme and becomes a product(s) that cooperates chemically with another substrate regarding formation of another product(s), a non-limitative example of which is an oxidant) with a relatively high oxidation potential (such as, for example, oxygen to hydrogen peroxide) or they may reduce the mediator M₍ᵣ₎, which is subsequently re-oxidized M(ox) by a working electrode 22 at a lower, more selective potential.

The activation of the apo-enzyme 20 by the fragment 16 and the subsequent reaction involving the holo-enzyme 24 results in the formation (or enhancement) of an optically and/or an electrochemically measurable enzymatic signal. It is to be understood that the optical and/or electrochemical measurement of the enzymatic signal corresponds to a measurement of the target DNA 17. The optical detection of GBS cfb gene using an embodiment of the method shown in FIG. 2A is shown in FIG. 2B. As depicted, the samples containing the target gene have a change in absorbance as the reaction involving the holo-enzyme takes place.

In this non-limitative example, a 0.2 ml PCR tube, about 3 μl (3.3*10+5) of GBS genomic DNA (ATCC BAA-611D), about 1 μl of 100 μM p-probe (a PQQ-probe), and about 1.0 μl of 100 μM primers were added to ingredients of the PCR mix (about 220 μM dNTP, about 1.65 mM MgCl₂, about 22 U Taq Polymerase/ml, about 55 mM KCl, and 22 mM Tris-HCl (pH 8.4), and stabilizers) to render a total reaction volume of about 50 μL. A hybridization primer-probe assay targeting the cfb gene that encodes the CAMP-factor protein was used. The thermal cycle run profile for the GBS cfb gene consists of a hot start of 1 cycle at about 94° C. for about 120 seconds, followed by amplification which includes 35 cycles at about 94° C. for about 15 seconds (i.e., denaturation occurs), exposure to about 55° C. for about 30 seconds for annealing, and exposure to about 72° C. for about 30 seconds for extension to occur. 1 cooling cycle was performed at about 68° C. for about 7 minutes. The PCR results were validated using conventional gel-electrophoresis method to identify the corresponding amplicons.

Post-PCR amplicons were transferred to a 96-well plate, to which assay reagents (about 30 μl 0.5 mM DCPIP, about 7.5 μL 20 mM CaCl₂, about 25 μL 80 mM glucose, and about 5 μL 57 μg/mL GDH) were added for the optical detection based upon apo-GDH-based signal amplification. DCPIP was used as a color indicator (λₘₐₓ=605 nm), which was reduced by the reconstituted holo-GDH. The absorbance was measured at 590 nm.

In FIG. 2B, PCR samples containing genomic GBS DNA showed a rapid decrease in the absorbance by the visual color distinction of DCPIP, whereas the controls (PCR samples missing any essential component) remained unchanged. λ-bacteriophage DNA (1.1*10⁺⁶) was used instead of GBS DNA as one of the controls. As there was no significant change in absorbance, these results substantially confirmed that the absorbance changes originated from the amplification of a target gene. Generally, the color change was highly conspicuous, and usually appeared at initial stages of the absorbance measurements. The triplicate data of the GBS DNA in FIG. 2B were easily differentiated in the time-sequential order of manual sample mixing and/or automated measurement (well to well time lag in scanned reading: approx. 0.8 sec).

Referring back to FIG. 2A, the embodiment illustrated is a homogeneous assay system. In such a homogeneous system, the intact labeled nucleotide 10 is substantially inactive. After the DNA amplification process, the mixture solution (the labeled nucleotide 10, fragment 16, the multiplied target DNA 17) will be mixed with the mediator (M) and/or a co-substrate (i.e. a reactant that is transiently associated with the enzyme and becomes a product(s) that cooperates chemically with another substrate regarding formation of another product(s), a non-limitative example of which is an oxidant), the apo-enzyme 20, nucleic acids, redox indicators and any other desired ingredients/reagents for optical and/or electrochemical detection, such as those described herein. In such a homogeneous system, the intact labeled nucleotide 10 may desirably have the tendency not to bind with the apo-enzyme 20, not to activate the holo-enzyme 24, and not to generate an optical and/or electrochemical signal that is representative of the DNA sample 17, as shown in FIG. 2B.

Referring back to the DNA amplification processes, in an embodiment using a PCR-free amplification process, the DNA amplification occurs without thermal denaturation of double stranded (ds) template DNA. By definition, the melting temperature of a given sample of DNA means the temperature at which half the population of dsDNA in the sample exists denatured, i.e. in a single-stranded form. This temperature is the inflection point of a sigmoidal melting curve of the given DNA sample. As such, a slight portion of dsDNA can be denatured and exist in a single stranded form at temperatures other than the melting temperature. In the presence of primers and probes 10 with appropriate sequences, the polymerase- and exonuclease-based amplification reaction may still occur at a relatively slow rate. In a non-limitative example embodiment, while the number of amplicons and PQQ released increases proportionally to the natural progression of the polymerase- and exonuclease-based amplification at room temperature (or at/near the melting temperature of primers and PQQ-probes), the activation of the enzyme (e.g. GDH) is highly pronounced, in part, because the product of this amplification reaction is theoretically capable of activating a single molecule (e.g. GDH) whose turnover number is around 10,000. Therefore, even without the thermocycling, an electrochemical or optical signal change indicating the existence of a gene sequence of interest may be generated.

In an embodiment using an end-point detection DNA amplification process, holo-enzyme 24 activity (which generates the enzymatic signal) is measured before and after the entire process. The methods for detection include, but are not limited to visual color change, absorbance change, fluorometry, electrochemistry, densitometry, voltammetry, amperometry, coulometry, potentiometry, conductivity, tensammetry, impedance measurements, and/or the like. It is to be understood that a sample designation as to whether “positive (with target DNA)” or “negative (without target DNA)” for a given DNA sequence may depend, at least in part, on a predetermined criterion involving the magnitude of the change in optical and/or electrochemical signal observed before and after the amplification process. In one embodiment using end-point detection, the PCR and detection processes may be performed sequentially as two separate steps in two separate and/or different spatial environments. In another embodiment using end-point detection, the PCR and optical and/or electrochemical detection may be batch processed in one integrated step in the same spatial environment. In a non-limitative example using batch processing, thermophilic or thermally stabilized enzymes (non-limitative examples of which include sol-gel and probe encapsulated by biologically localized embedding (PEBBLE)) may be used.

In an embodiment using a real-time detection DNA amplification process, holo-enzyme 24 activity (which generates the enzymatic signal) is measured continuously, or in many closely spaced (in time) discrete measurements, throughout the entire process. The optical and/or electrochemical signal may be detected using the methods previously described under oxidative or reductive conditions. It is to be understood that a sample designation as to whether “positive (with target DNA)” or “negative (without target DNA)” for a given DNA sequence may depend, at least in part, on a predetermined criterion involving the magnitude of the Delta comparing signal measurements before and after the PCR for each thermal cycle. In one embodiment using real-time detection, the PCR and detection processes may be performed sequentially as two separate steps in two separate and/or different spatial environments. In another embodiment using real-time detection, the PCR and optical and/or electrochemical detection may be batch processed in one integrated step in the same spatial environment. In a non-limitative example using batch processing, thermophilic or thermally stabilized enzymes (non-limitative examples of which include sol-gel and PEBBLE) may be used.

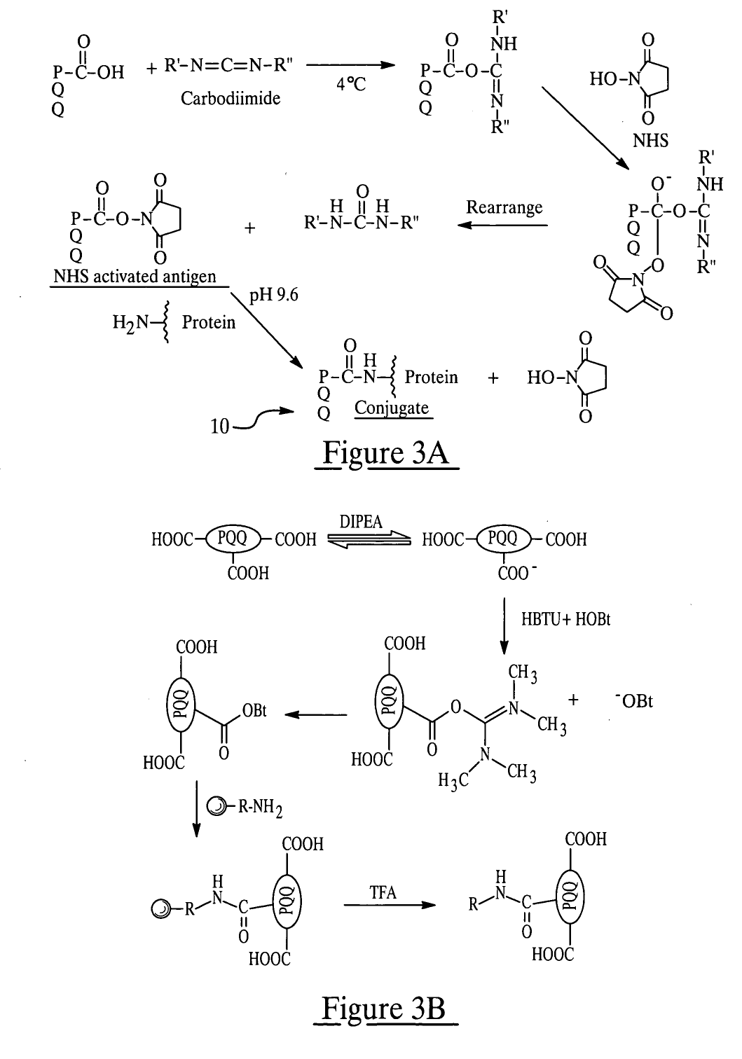

Referring now to FIG. 3A, an example embodiment of making a prosthetic group labeled protein (i.e. marker molecule) is schematically depicted. It is to be understood that the co-factor (CF) 14 (in this case PQQ) may be conjugated at any spot along the primary amine site in the protein sequence.

In the non-limitative example shown in FIG. 3A, the co-factor (CF) 14 (PQQ) is conjugated to the primary amine site at the end of the protein (a non-limitative example of which includes protamine) by adding 1-(3-dimethylaminopropyl)-3-ethylcarbodiimide (EDC or EDAC; 1 mg/ml) into a PQQ solution. After reacting the PQQ with EDAC at about 4° C., N-hydroxysuccinimide (NHS; 0.6 mg/ml) is injected into the solution and shaken. This results in the modification of the carboxyl groups and the formation of amine-reactive NHS esters. A solution of the desired protein may be added to the activated PQQ and allowed to react. The non-limitative example PQQ-protamine conjugate (marker molecule 10) shown in FIG. 3 is capable of monitoring peptide cleaving enzyme (e.g., trypsin) activity.

Referring now to FIG. 3B, another example embodiment of making a prosthetic group labeled peptide (i.e. marker molecule) is schematically depicted.

In the non-limitative example shown in FIG. 3B, the co-factor 14 (PQQ) is conjugated to the primary amine site at the end of a peptide (a non-limitative example of which includes HIV-protease). Synthetic peptides may be prepared using standard Fmoc (9-fluorenylmethoxycarbonyl) solid phase synthesis, and may be preserved in the form of a fully protected peptide having its N terminal free. PQQ is dissolved in DMF with slight sonication, and N-[(1H-benzotriazol[1[yl)(dimethylamino)methylene]-N-methylmethanaminium hexafluorophosphate N-oxide (HBTU) and 1-hydroxybenzotriazole (HOBt) are added into PQQ solution. The peptide resins are solvated in DMF for at least about 20 minutes. The activated PQQ may then be combined with the swollen resin peptides (where R denotes peptides with side chain protection groups). The reaction mixture may be shielded from ambient light and may be stirred for about 6 hours at room temperature. The peptide resins are filtered and washed with DMF, methanol, and deionized water. The peptide resins may then be dried and cleaved with freshly prepared trifluoroacetic (TFA) cleaving solution (where TFA:Triisopropylsilane:water=95:2.5:2.5 by volume). Resin beads are filtered off and residual TFA and organic solvents may be evaporated under a reduced pressure. The cleaved peptide-PQQ conjugates may be dissolved in deionized water.

This product may be subsequently purified using a reverse phase HPLC column. The non-limitative example the PQQ-peptide conjugate (marker molecule 10) shown in FIG. 3b is capable of monitoring peptide cleaving enzyme (e.g., trypsin) activity.

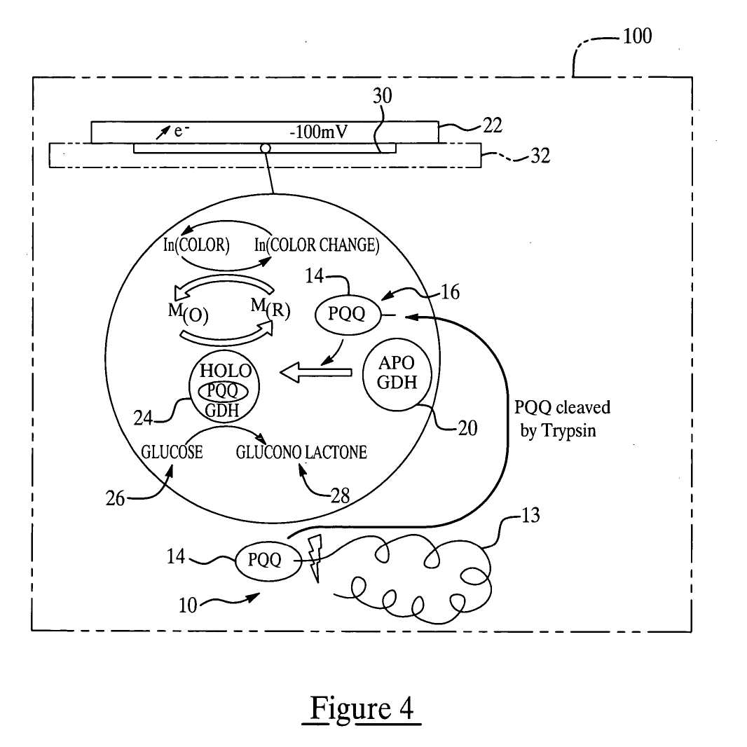

Referring now to FIG. 4, an embodiment of the diagnostic device 100 and a method of monitoring peptide cleaving enzyme (e.g., trypsin) activity in a sample using an embodiment of the marker molecule 10 is schematically depicted. Embodiment(s) of the method integrate cleaving processes with enzymatic signal amplification. More specifically, peptide cleaving enzyme (e.g., trypsin) activity may trigger enzymatic generation or amplification of a measurable optical and/or electrochemical enzymatic signal. The enzymatic signal(s) may be optically and/or electrochemically detectable via absorbance change, fluorescence, visual color change, electrochemistry, densitometry, voltammetry, amperometry, coulometry, potentiometry, conductivity, tensammetry, impedance measurements, and/or combinations thereof.

Embodiment(s) of the method generally include exposing an embodiment of the co-factor labeled protein, peptide, or oligonucleotide 10 to cleaving activity (e.g. peptide, protein, or nucleotide cleaving activity), combining a co-factor (CF) 14 fragment 16 to an apo-enzyme 20, and optically or electrochemically detecting an enzymatic signal that results from the combination of the fragment 16 with an apo-enzyme 20. It is to be understood that these steps may be performed substantially simultaneously or sequentially.

Cleaving enzyme activity processes may include formulating a sample such that it is compatible with desired chemistries for enzymatic signal amplification and optical and/or electrochemical detection. Such a formulation may include, but is not limited to the following: substrate(s) (a non-limitative example of which is glucose), marker molecules 10 (e.g. PQQ-protamine conjugate(s)), buffers (non-limitative examples of which include Tris, HEPES, phosphate, and the like), mediators (non-limitative examples of which include ferricyanide, ferrocene derivatives, phenazine methosulfate (PMS), Ru(III) complexes, ubiquinone (Qo), Os complexes, and the like), stabilizers (non-limitative examples of which include CaCl₂, MgCl₂, and the like), redox inhibitors (dichloroindolephenol (DCIP), resazurin, thionine, and the like), enzyme thermal stabilizers, barriers, oligo binders, and/or mixtures thereof.

As depicted in FIG. 4, the intact marker molecule 10 includes the protein (e.g., protamine) 13 having PQQ as the co-factor (CF) 14 conjugated thereto. Cleaving enzyme (e.g., trypsin) activity results in the hydrolysis of the marker molecule 10. The embodiment depicted in FIG. 4, and in particular the exploded portion, is an assay system in which the test strip 22 is coated with a film 30 including the mediator (or co-substrate), the indicator, and the apo-enzyme 20 (e.g. apo-GDH), which is physically separated from the solution containing the marker molecule 10 via an optional film 32.

The hydrolysis of the marker molecule 10 by peptide cleaving enzyme (e.g., trypsin) releases (as depicted by the lightening bolt) a fragment 16 containing the co-factor (CF) 14. The fragment 16 containing the co-factor (CF) 14 may then combine with and activate an apo-enzyme 20 immobilized on the surface of a test strip 22 or a working electrode. It is to be understood that the apo-enzyme 20 may also be present in solution when the assay is homogeneous. The combination of the fragment 16 and the apo-enzyme 20 forms a holo-enzyme 24, which is capable of catalyzing a reaction that converts a predetermined substrate 26 in the sample to a product 28 plus free electrons. These free electrons may reduce a mediator M(R), which is subsequently re-oxidized M(OX) by a redox indicator (In(color)) (a non-limitative example of which includes a dye) that results in the color change of the redox indicator (In(color change)). The free electrons may also reduce a co-substrate with a relatively high oxidation potential (such as, for example, oxygen to hydrogen peroxide) or they may reduce the mediator M(r), which is subsequently re-oxidized M(ox) by a working electrode at a lower, more selective potential.

The activation of the apo-enzyme 20 by the fragment 16 and the subsequent reaction involving the holo-enzyme 24 results in the formation or amplification of an optically and/or electrochemically measurable enzymatic signal. It is to be understood that the optical and/or electrochemical measurement of the enzymatic signal corresponds to monitoring cleaving enzyme activity.

The embodiment shown in FIG. 4 is a homogeneous assay system. In such a homogeneous system, the intact marker molecule 10 is substantially inactive. After or during the cleaving process, the mixture solution (the PQQ-protamine conjugate 10 and fragment 16) will be mixed with the mediator (M) and/or a co-substrate (i.e. a reactant that is transiently associated with the enzyme and becomes a product(s) that cooperates chemically with another substrate regarding formation of another product(s), a non-limitative example of which is an oxidant), the apo-enzyme 20, redox indicators and any other desired ingredients/reagents for optical detection, such as those described herein. In such a homogeneous system, the intact marker molecule 10 may desirably have the tendency not to bind with the apo-enzyme 20, not to activate the holo-enzyme 24, and to not generate an optical signal that is representative of the cleaving enzyme.

Referring back to the cleaving processes, holo-enzyme 24 activity (which generates the enzymatic signal) is measured through the entire process in real time. The methods for detection include, but are not limited to visual color change, absorbance change, fluorometry, electrochemistry (potentiometry, amperometry, voltametry, etc.), and the like. In a non-limitative example using batch processing, thermophilic or thermally stabilized enzymes (non-limitative examples of which include sol-gel and probe encapsulated by biologically localized embedding (PEBBLE)) may be used.

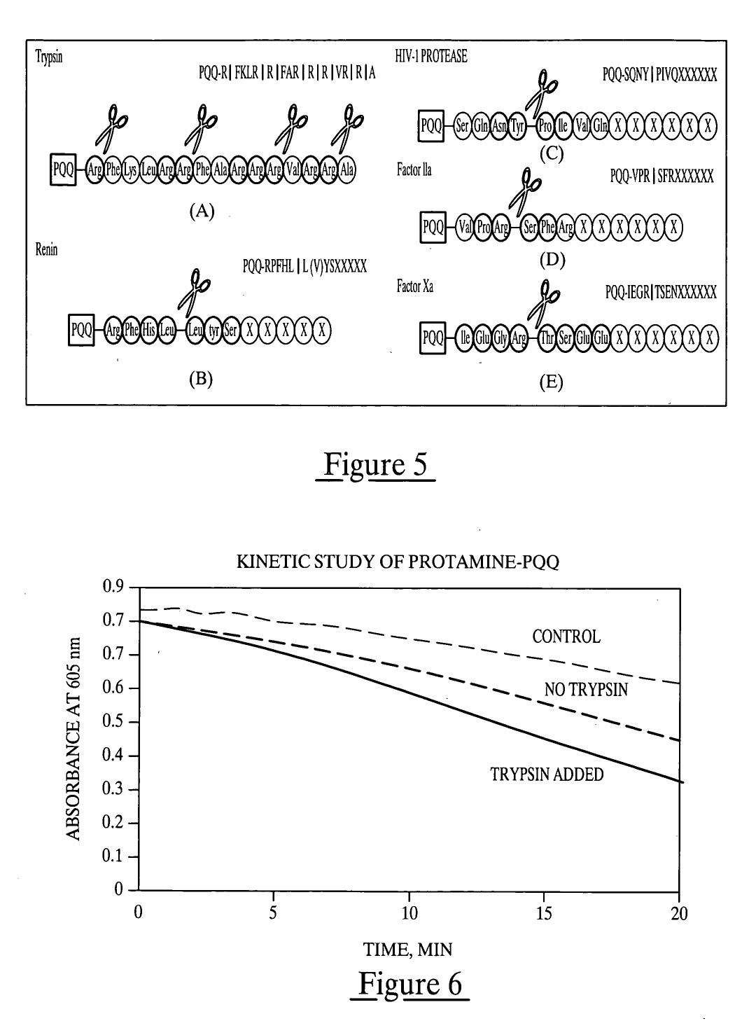

FIG. 5 depicts non-limitative examples of various probes 10 that have specific cleaving sites (as depicted by the scissors) for target enzymes. The probe 10 designated (A) is a PQQ-labeled peptide probe having four cleaving sites for the target enzyme trypsin. The probe 10 designated (B) is a PQQ-labeled peptide probe having a cleaving site for the target enzyme renin (PQQ Arg Pro Phe His Leu Leu(Val) Tyr Ser Glu Ala Glu Ala Val Phe Val Phe Val Phe Leu Phe Val Phe Val Phe Leu). The probe 10 designated (C) is a PQQ-labeled peptide probe having a cleaving site for the target enzyme HIV-1 protease (PQQ Ser Gln Asn Tyr Pro Ile Val Gln Glu Ala Glu Ala Val Phe Val Phe Val Phe Leu Phe Val Phe Val Phe Leu). The probe 10 designated (D) is a PQQ-labeled peptide probe having a cleaving site for the target enzyme Factor IIa (i.e. thrombin or FlIa) (PQQ Val Pro Arg Ser Phe Arg Asn Ala Glu Ala Glu Ala Val Phe Val Phe Val Phe Leu Phe Val Phe Val Phe Leu). The probe 10 designated (E) is a PQQ-labeled peptide probe having a cleaving site for the target enzyme Factor Xa (FXa;) (PQQ Ile Glu Gly Arg Thr Ser Glu Asn Glu Ala Glu Ala Val Phe Val Phe Val Phe Leu Phe Val Phe Val Phe Leu). It is to be understood that the “X” in probes B, C, D and E in FIG. 5 represent any hydrophobic amino acid.

It is to be understood that the embodiments of the probe 10 disclosed herein may be chimeric probes including a linker molecule positioned between the co-factor 14 and the peptide, protein, or oligonucleotide. Non-limitative examples of such linker molecules include PQQ-peptide-oligonucleotide probe, PQQ-peptide-PNA oligomer probe, and PQQ-peptide-DNA oligomer probe. It is to be understood that the chimeric probe may recognize the target DNA sequence and may release the probe fragment 16 by protease action on the linker molecule. The fragment 16 activates the apo-enzyme 20 to produce a signal change capable of assaying the target DNA sequence qualitatively and/or quantitatively. It is to be further understood that the linker molecule may be any molecule capable of being cleaved by a chemical or biological reaction, such that the probe fragment 16 is generated to activate the apo-enzyme 20. In a non-limitative example, the linker is a peptide.

Experimental

Homogeneous Optical and Electrochemical Assay for Trypsin activity with GDH and PQQ-Protamine Marker Molecule

The PQQ-protamine marker molecule (4 nM) solutions included APO-GDH (1 μM), 0.2mM CaCl₂, and 40 mM Glucose. 60 μM PMS and a redox indicator (0.3 mM DCIP) were also added. (See FIG. 6)

Homogeneous Optical and Electrochemical Assay for Trypsin activity with GDH and PQQ-Peptide Marker Molecule

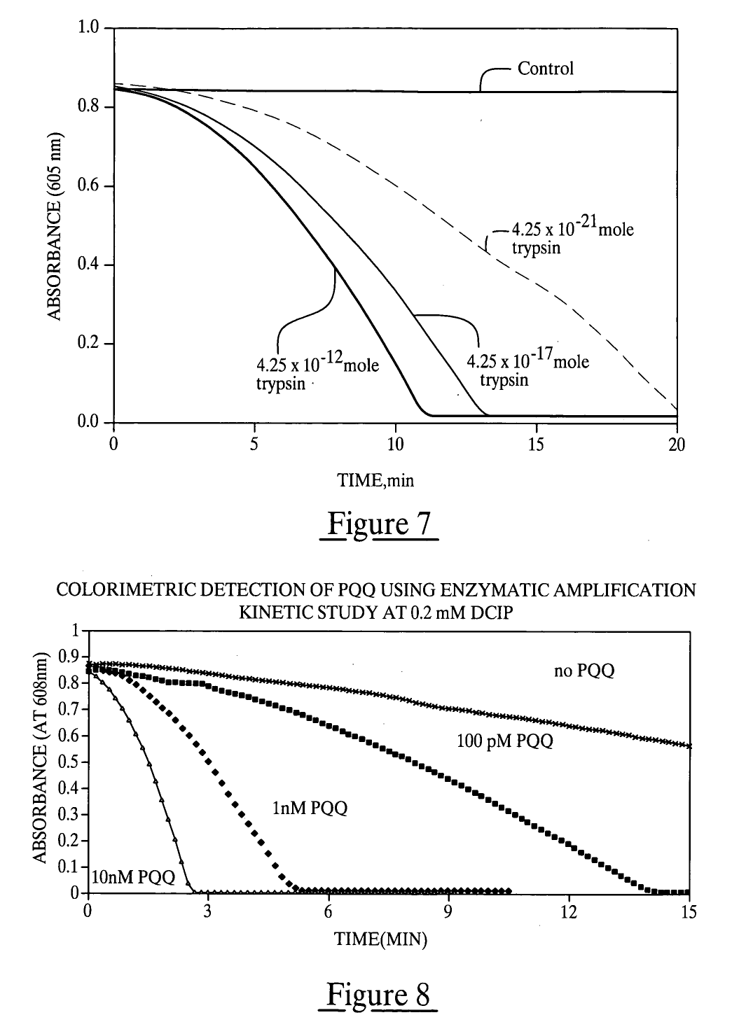

The PQQ-peptide marker molecule (2 nM) solutions included APO-GDH (2 nM), 0.2mM CaCl₂, and 40 mM Glucose. 60 μM PMS and a redox indicator (0.3 mM DCIP) were also added. (See FIG. 7.)

Homogeneous Optical and Electrochemical Assay for DNA with GDH and PQQ (See FIG. 8)

The PQQ solutions (variable concentrations) included APO-GDH (1 μM), 2 mM CaCl₂, and 40 mM Glucose. 50 μM PMS and a redox indicator (0.1-0.2 mM DCIP, 0.025 mM resazurin, or 0.05 mM thionine) were also added. PQQ at 0.1 nM was detectable.

Homogeneous Optical and Electrochemical Assay for DNase activity with PQQ-Oligonucleotide Marker Molecule

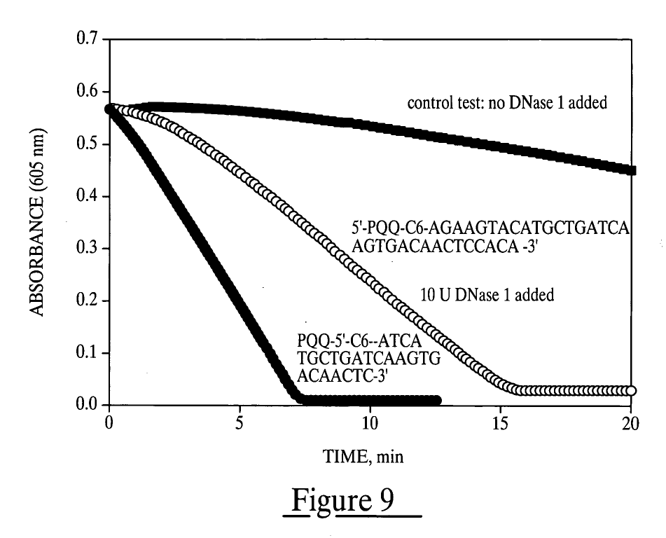

The PQQ-oligonucleotide marker molecule (10 nM) solutions included APO-GDH (1 μM), 0.2 mM CaCl₂, and 40 mM Glucose. 60 μM PMS and a redox indicator (0.3 mM DCIP) were also added. (See FIG. 9)

While several embodiments have been described in detail, it will be apparent to those skilled in the art that the disclosed embodiments may be modified. Therefore, the foregoing description is to be considered exemplary rather than limiting.

What is claimed is:

1. A marker molecule for monitoring cleaving enzyme activity, the marker molecule comprising:
a protein, a peptide, or an oligonucleotide; and
a co-factor conjugated to the protein, the peptide, or the oligonucleotide, thereby forming a co-factor labeled protein, a co-factor labeled peptide, or a co-factor labeled oligonucleotide;
wherein the co-factor is adapted to produce an enzymatic signal that is at least one of electrochemically and optically detectable.

2. The marker molecule as defined in claim 1 wherein the co-factor is one of a prosthetic group, a co-enzyme, and a metal-ion activator.

3. The marker molecule as defined in claim 1 wherein the co-factor is one of pyrroloquinoline quinone, flavin adenine dinucleotide, nicotinamide adenine dinucleotide phosphate, heme, phenazine methosulfate, phenazine ethosulfate, naphthoquinone, derivatives thereof, benzoquinone, derivatives thereof, fluorescein, derivatives thereof, thionine, resazurin, 2,6-dichlorphenoleindophenole, orthoquinone, and derivatives thereof.

4. The marker molecule as defined in claim 1 wherein the enzymatic signal is optically detectable via absorbance change, fluorescence, visual color change, electrochemistry, densitometry, and combinations thereof.

5. The marker molecule as defined in claim 1 wherein the enzymatic signal is electrochemically detectable via at least one of voltammetry, amperometry, coulometry, potentiometry, conductivity, tensammetry, impedance measurements, or combinations thereof.

6. The marker molecule as defined in claim 1 wherein the co-factor is adapted to release from the labeled protein, the labeled peptide, or the labeled oligonucleotide and to bind to and activate an apo-enzyme, thereby forming a holo-enzyme that is adapted to catalyze a reaction to produce electrons.

7. The marker molecule as defined in claim 1 wherein the co-factor labeled protein, the co-factor labeled peptide, or the co-factor labeled oligonucleotide further comprises a linker molecule between the co-factor and the protein, the peptide, or the oligonucleotide.

8. A labeled oligonucleotide for enzyme amplified target DNA detection, the labeled oligonucleotide comprising:
a site-specific sequence; and
a co-factor conjugated to the site-specific sequence;
wherein the co-factor is adapted to produce an enzymatic signal that is at least one of electrochemically and optically detectable.

9. The labeled oligonucleotide as defined in claim 8 wherein the co-factor is one of a prosthetic group, a co-enzyme, and a metal-ion activator.

10. The labeled oligonucleotide as defined in claim 8 wherein the co-factor is one of pyrroloquinoline quinone, flavin adenine dinucleotide, nicotinamide adenine dinucleotide phosphate, heme, phenazine methosulfate, phenazine ethosulfate, naphthoquinone, derivatives thereof, benzoquinone, derivatives thereof, fluorescein, derivatives thereof, thionine, resazurin, 2,6-dichlorphenoleindophenole, orthoquinone, and derivatives thereof.

11. The labeled oligonucleotide as defined in claim 8 wherein the site specific sequence is 5′-amino-X—PO₄-CCAAAAGGTACACCTGTTTGAG-3′, with X selected from —(CH₂)ₙ—, —(CH₂)ₒO(CH₂)ₚ—, and —(CH₂)_{q}S(CH₂)ᵣ—, with n selected from the numbers two through twelve, and with o, p, q, and r selected from the numbers two through six, wherein the co-factor is pyrroloquinoline quinone, and wherein the target DNA is from P. pachyrhizi.

12. The labeled oligonucleotide as defined in claim 8 wherein the enzymatic signal is optically detectable via absorbance change, fluorescence, visual color change, electrochemistry, densitometry, and combinations thereof.

13. The labeled oligonucleotide as defined in claim 8 wherein the enzymatic signal is electrochemically detectable via at least one of voltammetry, amperometry, coulometry, potentiometry, conductivity, tensammetry, impedance measurements, or combinations thereof.

14. The labeled oligonucleotide as defined in claim 8 wherein the co-factor is adapted to release from the site-specific sequence and to bind to and activate an apo-enzyme, thereby forming a holo-enzyme that is adapted to catalyze a reaction to produce electrons.

15. The labeled oligonucleotide as defined in claim 1, further comprising a linker attaching the co-factor to the protein, the peptide, or the oligonucleotide.

16. A method of detecting target DNA in a sample, the method comprising:
performing a DNA amplification process on the sample;
exposing a labeled oligonucleotide to exonuclease activity, the labeled oligonucleotide including a site-specific sequence and a co-factor conjugated to the site-specific sequence, the exposing thereby releasing a fragment including the co-factor;
combining the fragment with an apo-enzyme, wherein combining the fragment including the co-factor with the apo-enzyme produces an enzymatic signal that is at least one of electrochemically and optically detectable; and
at least one of electrochemically and optically detecting the enzymatic signal, thereby detecting the target DNA.

17. The method as defined in claim 16 wherein the DNA amplification process is at least one of real time PCR, end-point PCR, PCR-free amplification, rolling cycle amplification, isothermal based amplification methods, thermocycling based amplification methods, or combinations thereof.

18. The method as defined in claim 17 wherein the DNA amplification process is real time PCR and wherein the enzymatic signal is detected at predetermined intervals during the amplification process.

19. The method as defined in claim 17 wherein the DNA amplification process is end-point PCR and wherein the enzymatic signal is detected prior to and after the amplification process.

20. The method as defined in claim 17 wherein the DNA amplification process is PCR-free amplification, and wherein the enzymatic signal is detected at least one of prior to, at predetermined intervals during, or after the amplification process.

21. The method as defined in claim 16 wherein the DNA amplification process includes exposing the sample to a PCR mixture including at least one of substrates, probes, buffers, mediators, stabilizers, redox indicators, calcium chloride, magnesium chloride, or combinations thereof.

22. The method as defined in claim 16 wherein the performing, exposing, combining, and detecting occur one of substantially simultaneously and sequentially.

23. The method as defined in claim 16 wherein optically detecting the enzymatic signal is accomplished via absorbance change, fluorescence, visual color change, electrochemistry, densitometry, and combinations thereof.

24. The method as defined in claim 16 wherein electrochemically detecting the enzymatic signal is accomplished via at least one of voltammetry, amperometry, coulometry, potentiometry, conductivity, tensammetry, impedance measurements, or combinations thereof.

25. A method of monitoring cleaving enzyme activity in a sample, the method comprising:
exposing a co-factor labeled protein, peptide, or oligonucleotide to cleaving activity, the co-factor labeled protein, peptide, or nucleotide including a protein, peptide, or nucloetide and a co-factor conjugated to the protein, peptide, or nucleotide, the exposing thereby releasing a fragment including the co-factor;
combining the fragment with an apo-enzyme, wherein combining the fragment including the co-factor with the apo-enzyme produces an enzymatic signal that is at least one of electrochemically and optically detectable; and
at least one of electrochemically and optically detecting the enzymatic signal, thereby monitoring cleaving enzyme activity.

26. The method as defined in claim 25 wherein optically detecting the enzymatic signal may be accomplished via at least one of absorbance change, fluorescence, visual color change, electrochemistry, densitometry, or combinations thereof.

27. The method as defined in claim 25 wherein electrochemically detecting the enzymatic signal is accomplished via at least one of voltammetry, amperometry, coulometry, potentiometry, conductivity, tensammetry, impedance measurements, or combinations thereof.

28. The method as defined in claim 25 wherein the co-factor is one of a prosthetic group, a co-enzyme, or a metal-ion activator.

29. The method as defined in claim 28 wherein the co-factor is one of pyrroloquinoline quinone, flavin adenine dinucleotide, nicotinamide adenine dinucleotide phosphate, heme, phenazine methosulfate, phenazine ethosulfate, naphthoquinone, derivatives thereof, benzoquinone, derivatives thereof, fluorescein, derivatives thereof, thionine, resazurin, 2,6-dichlorphenoleindophenole, orthoquinone, and derivatives thereof.

30. The method as defined in claim 25 wherein the cleaving activity is peptide cleaving activity, nucleotide cleaving activity, or protein cleaving activity.

31. The method as defined in claim 25 wherein the cleaving activity is accomplished via a cleaving enzyme.

32. The method as defined in claim 31 wherein the cleaving enzyme is selected from DNases, polymerases, proteases, renin, FVa, FXa, FIIa, and HIV-protease.

33. The method as defined in claim 25 wherein the cleaving enzyme activity is adapted to monitor an anticoagulation effect of an anticoagulation reagent, to measure activated clotting time, to measure activated partial thromboplastic time, to measure thrombin time, or combinations thereof.

34. An optical diagnostic device for detecting target DNA, the device comprising:
a multi-well plate having a microplate reader; and
a reaction mixture disposed within the multi-well plate, the reaction mixture including an apo-enzyme and a labeled oligonucleotide, the labeled oligonucleotide including:
a site-specific sequence; and
a co-factor conjugated to the site-specific sequence;
wherein the co-factor is cleaveable from the labeled oligonucleotide to form a probe fragment that binds to the apo-enzyme to produce an enzymatic signal that is optically detectable.

35. The optical diagnostic device as defined in claim 34 wherein the reaction mixture further includes at least one of enzyme substrates, buffers, mediators, stabilizers, redox indicators, color indicators, calcium chloride, magnesium chloride, or combinations thereof.

36. The optical diagnostic device as defined in claim 34 wherein the enzymatic signal is optically detectable via absorbance change, fluorescence, visual color change, electrochemistry, densitometry, and combinations thereof.

37. An optical diagnostic device for optically monitoring cleaving enzyme activity, the device comprising:
a multi-well plate having a microplate reader; and
a reaction mixture disposed within the multi-well plate, the reaction mixture including an apo-enzyme and a marker molecule, the marker molecule including:
a protein, a peptide, or an oligonucleotide; and
a co-factor conjugated to the protein, the peptide, or the oligonucleotide, thereby forming a co-factor labeled protein, a co-factor labeled peptide, or a co-factor labeled oligonucleotide;
wherein the co-factor is cleaveable from the protein, the peptide, or the oligonucleotide and is able to bind to and activate the apo-enzyme to produce an enzymatic signal that is optically detectable.

38. The optical diagnostic device as defined in claim 37 wherein the reaction mixture further includes at least one of enzyme substrates, buffers, mediators, stabilizers, redox indicators, color indicators, calcium chloride, magnesium chloride, or combinations thereof.

39. The optical diagnostic device as defined in claim 37 wherein the enzymatic signal is optically detectable via absorbance change, fluorescence, visual color change, electrochemistry, densitometry, and combinations thereof.

40. An electrochemical diagnostic device for detecting target DNA, the device comprising:
at least one electrode having an apo-enzyme immobilized thereon; and
a labeled oligonucleotide in electrochemical contact with the at least one electrode, the labeled oligonucleotide including:
a site-specific sequence; and
a co-factor conjugated to the site-specific sequence;
wherein the co-factor is cleaveable from the labeled oligonucleotide to form a probe fragment that binds to the apo-enzyme to produce an enzymatic signal that is electrochemically detectable.

41. The electrochemical diagnostic device as defined in claim 40 wherein the enzymatic signal is electrochemically detectable via at least one of voltammetry, amperometry, coulometry, potentiometry, conductivity, tensammetry, impedance measurements, or combinations thereof.

42. An electrochemical diagnostic device for electrochemically monitoring cleaving enzyme activity, the device comprising:
at least one electrode having an apo-enzyme immobilized thereon; and
a marker molecule in electrochemical contact with the at least one electrode, the marker molecule including:
a protein, a peptide, or an oligonucleotide; and
a co-factor conjugated to the protein, the peptide, or the oligonucleotide,
wherein the co-factor is cleaveable from the protein, the peptide, or the oligonucleotide and is able to bind to and activate the apo-enzyme to produce an enzymatic signal that is electrochemically detectable.

43. The electrochemical diagnostic device as defined in claim 42 wherein the enzymatic signal is electrochemically detectable via at least one of voltammetry, amperometry, coulometry, potentiometry, conductivity, tensammetry, impedance measurements, or combinations thereof.

44. A labeled oligonucleotide for enzyme amplified target DNA detection, the labeled oligonucleotide comprising:
a site-specific sequence;
a co-factor; and
a linker conjugating the co-factor to the site-specific sequence;
wherein the linker is cleaveable, thereby forming a co-factor fragment that is adapted to produce an enzymatic signal that is at least one of electrochemically and optically detectable.
